(19) United States
(12) Plant Patent Application Publication
(10) Pub. No.: US 2011/0035853 P1
(43) Pub. Date: Feb. 10, 2011

(54) Distinct cultivar of Hosta plant named 'Foxfire Night Skye'

(50) ;

(76) Inventor: Stephen John Schulte, Marshfield, WI (US)

Correspondence Address: Stephen J. Schulte, 28 Dalton Street, Asheville, NC, 28803, US

(21) Appl. No.: 12/462,652

(22) Filed: Aug. 7, 2009

(51) Int. Cl. A01H 5/00

(52) U.S. Cl. PLT/353

(57) ABSTRACT

A distinct mutation selection of Hosta ‘Edge of Night’ (not patented) named Hosta ‘Foxfire Night Skye’ exhibiting dark green leaves with a wide creamy margin, striking contrast and vigorous growth rate.

### CROSS REFERENCE TO RELATED APPLICATIONS

None

### STATEMENT REGARDING FEDERALLY SPONSERED R&D

None

### LATIN NAME OF GENUS AND SPECIES OF CLAIMED PLANT

Genus: Hosta

Species: Not routinely designated in this plant group as cross hybridizing often obscures the ‘species parentage’ and makes it impossible to clearly determine. Species designation may occur (always written in lower case italics) if the claimed plant were a direct mutation or selection of one of the thirty-three or more actual recognized species. The plant Hosta sieboldiana ‘Elegans’ would represent an example of this. This plant is a selection of a superior form of the species sieboldiana and is known in the market by its cultivar designation of ‘Elegans’. Any offspring resulting in a cross of sieboldiana ‘Elegans’ with another hosta though, would probably no longer carry the species designation. Such an offspring, as most all hostas, would simply be designated as Hosta ‘cultivar name’.

### VARIETY DENOMINATION

‘Foxfire Night Skye’ is the variety designation and the name that will be used in the landscape and container growing market trade.

### BACKGROUND OF THE INVENTION

The present invention relates to a new and distinct cultivar of Hosta plant, botanically known as Hosta hybrid, and hereinafter referred to by the cultivar name ‘Foxfire Night Skye’.

The new cultivar was discovered in Marshfield, Wis. by the inventor as a naturally occurring leaf mutation of a plant of the nonpatented Hosta Hybrid cultivar ‘Edge of Night’ in 2004. The parent cultivar is characterized by a medium to large foliage mound 48 inches wide by 24 inches tall. The leaves are broadly ovate and arching, 8 inches wide and 9 inches long. They are dark green in color. Petioles are green in color and flowers are described as lavender and tubular. This parent cultivar was produced by a cross between two breeding cultivars by Robert Savory designated as H. ‘Green Gold’×H. ‘Halcyon’ and registered in 1988. (not patented)

The foliage mound of the new Hosta, ‘Foxfire Night Skye’, is similar to its parent in size (48 inches (122 cm) wide by 24 inches (62 cm) high). The leaves are large (8 inches (20 cm) wide by 9 inches (23 cm) long) and dark green in color. The new plant is distinguished from its parent by a wide cream margin that encircles the leaf ranging in size from one-half inch (13 mm) in thickness to one and one half inch (39 mm). Lighter shades of green and yellow flare from the outer edge of the dark green center into the adjoining edge of the white margin. Subsequent asexual propagation by division at Foxfire Botanical Gardens in 2007 as well as tissue culture in 2008 at Conyers, Ga. has shown the unique and distinct characteristics of this new plant are stable and reproduce true to type in successive generations.

### SUMMARY OF THE INVENTION

The following traits have been repeatedly observed and are determined to be the unique characteristics of ‘Foxfire Night Skye’ as a new and distinct cultivar.

1. Medium to large sized clump.

2. Dark green rounded leaves with wide, creamy, somewhat irregular margins approximately one-half to one and one-half inches in width characterized further by irregular light green and yellow streaking from the dark green center into the cream margin.

3. Striking contrast between the very dark green center and bright margin

4. Vigorous growth

The new Hosta can be compared to its all green parent cultivar, H. ‘Edge of Night’. Plants of the new Hosta have a distinct irregular cream margin enhanced with light green and yellow streaking between the green center and white border.

### BRIEF DESCRIPTION OF THE DRAWING

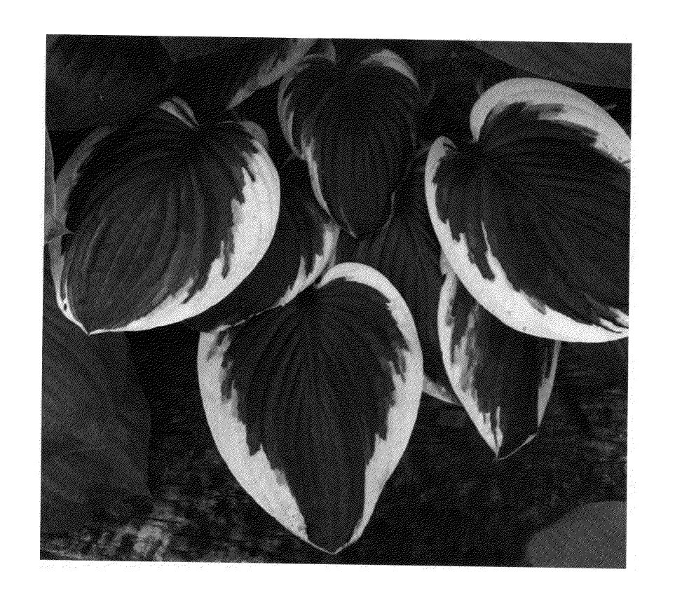

The accompanying color photograph (FIG. 1.) illustrates the overall appearance of the new cultivar, showing the colors as true as it is reasonably possible to obtain in colored reproductions of this type. The photograph comprises a top perspective view of a plant “Foxfire Night Skye” in a cultivated area of the garden. There may be slight differences in the leaf colors of the photograph from the actual colors due to light reflectance.

### DETAILED BOTANICAL DESCRIPTION

In the following description, color references are made to The Royal Horticultural Society Colour chart except where general terms of ordinary dictionary significance are used. The following observations and measurements describe the plant as grown in the ground in Central Wisconsin under partial shade.
Botanical classification: Hosta hybrid cultivar ‘Foxfire Night Skye’
Parentage: Mutant of Hosta ‘Edge of Night’
General description: a) Blooming period about 4 weeks from July 10^{th} to August 8^{th} in Wisconsin b) Plant habit: Herbaceous perennial, clump forming, mounded habit d) Height and spread reaches about 24 inches (62 cm) high by 48 inches (122 cm) wide e) Hardiness: USDA Zone 3-8 f) Diseases and pests: No superior or inferior susceptibility or resistance to diseases of other pests has been observed for Hosta ‘Foxfire Night Skye’ g) Culture: Light shade to partial sun, moist soils of moderate fertility h) Root description: Freely branched, fleshy i) Propagation” In vitro propagation is preferred using typical methods for Hosta, division is also possible j) Root development: Rooted transplants from tissue culture fully develop in a 72-cell liner in about 5-9 weeks in a greenhouse with average temperature of about 70 degrees F. k) Growth Rate: Vigorous
Foliage description: a) Leaf shape: broadly ovate b) Leaf Base: cordate c) Leaf size: 8 inches (20 cm) wide by 9 inches (23 cm) long d) Leaf quantity: 4-5 per shoot (eye) e) Leaf appearance: lightly wavy, smooth edge f) Leaf surface: top surface slightly shiny bottom surface: glaucous bloom g) Leaf venation: About 10 to 11 pairs of veins, campylodrome pattern, impressed on upper surface and raised on lower surface, color matches foliage coloration h) Leaf margin: smooth, slightly wavy i) Leaf arrangement: Basal, radiate spirally from base Leaf color: (July 20^{th}, 50% shade, Wisconsin) Dark green (nearest to RHS 137A) with wide, creamy (nearest to RHS 152D, (early spring) and then lightening to near RHS 155D as the season progresses), somewhat irregular margins, approximately 0.5 inches (13 mm) to 1.5 inches (39 mm) in width characterized further by irregular light green (nearest RHS 194A) and yellow (nearest RHS j) 145B) streaking from the dark green center into the cream margin. Margin color lightens as season progresses. k) Petiole Description: a) About 15 cm, 2-3 cm in width b) Petiole color: Central region is green (about RHS 144A) c) Petiole surface glaucous d) Petiole shape: Sulcate
Flower scape description: a) Scape shape: round, solid b) Scape Number: One per mature eye c) Scape posture: Straight, upright, some arching d) Scape size: 60 cm long 5-7 mm wide e) Scape Color: green (about RHS 146C f) Scape surface glaucous g) Leaf bracts: about 15-25 per scape, 25 mm length, 15 mm width, rigid, lanceolate in shape Dull on upper surface (about RHS 137C), shiny on lower surface (about RHS 137A), very narrow white edge.
Flower type: a) Inflorescence Type: Terminal racemes of single tubular-shaped flowers, closely spaced on the last 10 cm of the scape. b) Lastingness of inflorescence — about 4 weeks from first opening bloom to fading of last opening bloom, individual blooms lasting about one day c) Flower number: 15-25 d) Flower fragrance: none detected e) Flower Shape: tubular f) Flower bud shape: Spathulate g) Flower bud color: Light lavender (near RHS 85B) h) Flower size: 6 cm long, 1.5 cm wide i) Flower color: White (about RHS 155D) with lavender striations (about RHS 85B on inner surface j) Pedicel: 5 mm in length, 2 mm in diameter, (about RHS 145C in color) k) Perianth features: Comprised of six tepals, 3 internal, 3 external, overlapping in expanded region and fused in tube region l) Tepal Shape: Oblanceolate with acute apex m) Tepal size 6 cm in length and 1.5 cm in width. n) Tepal Color: white (about RHS N155D) with lavender striations (about RHS 85B) on the inner surface o) Tepal Texture: Glaucous
Reproductive organs:
Gynoecium: a) 1 pistil; style is about 4 cm in length and 1 mm in diameter, white in color; stigma is 3 lobed and white in color; ovary is superior, compound, composed of 3 locules, green (about RHS 145A) in color
Androecium: 6 stamens; filament is about 4 cm in length and 1 mm in diameter, white in color, pollen is abundant and about RHS 15B in color Fruit: Not observed under growing conditions tested Seeds Not observed under growing conditions tested.

I claim:

1. A new and distinct cultivar of ornamental Hosta plant named ‘Foxfire Night Skye’, substantially herein described and shown, having wide, creamy leaf margins with interjecting streaks of light green and yellow into the dark green center, suitable for the garden, cut flowers and potted plants.
